(19) United States
(12) Patent Application Publication
(10) Pub. No.: US 2006/0258987 A1
(43) Pub. Date: Nov. 16, 2006

(54) Catheter stiffening member

(75) Inventors: D. Christian Lentz, Bloomington, IN (US); Darin G. Schaeffer, Bloomington, IN (US); Kimberly D. Roberts, Bloomfield, IN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Incorporated, Bloomington, IN (US)

(21) Appl. No.: 11/429,845

(22) Filed: May 8, 2006

Related U.S. Application Data
(60) Provisional application No. 60/679,630, filed on May 10, 2005.

(51) Int. Cl. A61M 5/178

(52) U.S. Cl. 604/164.01

(57) ABSTRACT

A stiffening member for providing controllably variable stiffness to a catheter. A hollow elongated tubular member is sized to be received in a lumen of the catheter. At least a length of the tubular member is configured, such as by forming a spiral cut therethrough, in a manner such that the distal portion of the tubular member has a greater flexibility than the proximal portion.

### RELATED APPLICATION

The present patent document claims the benefit of the filing date under 35 U.S.C. §119(e) of Provisional U.S. Patent Application Ser. No. 60/679,630, filed May 10, 2005, which is hereby incorporated by reference.

### BACKGROUND

1. Technical Field

The present invention relates to a stiffening member for a medical device, such as a catheter. More particularly, the invention relates to a stiffening member having a variable stiffness along at least a portion of its length.

2. Background Information

Catheters are in widespread use in many aspects of modem medicine. They are typically percutaneously introduced into the vasculature of a patient over a wire guide that has been previously inserted using, for example, the well-known Seldinger technique. Catheters are commonly used for introducing a medical interventional device, such as a stent, into a desired area of the vasculature, and for introducing a liquid medicament into the vascular system of a patient.

In order to properly advance a catheter to a desired portion of the vasculature, it is often necessary to thread or otherwise force the catheter through increasingly narrow spaces within the confines of the vasculature. In addition, the pathway for the catheter may become increasingly tortuous, as the pathway may include a series of sharp angles or bends which must be navigated by the catheter as it is inserted deeper into the vasculature. Frequently, a catheter will kink as it traverses these narrow and/or tortuous spaces. A kinked catheter resists proper placement and is generally useless. As a result, the kinked catheter must be removed and replaced with another catheter. In addition to the foregoing, a catheter may also be required to traverse an obstruction, or stenosis, that is disposed in the vasculature along the pathway. The catheter may lack sufficient strength, or torque, to pass through the obstruction. Further advancement of a catheter may become problematic as a result of any of these factors, as well as a myriad of other possible complications that may be encountered.

Many techniques have been developed to assist the physician in properly introducing a catheter or other medical device into a desired portion of the vasculature. For example, many catheters are provided with a reinforcement member, such as a helical coil or a braid, that is incorporated into the wall structure of the catheter. A coil is particularly useful for enhancing the kink resistance of a catheter, while a braid is particularly useful for enhancing the pushability, or “torqueability”, of the catheter. While each of these reinforcements is generally effective for its intended purpose, each also has its shortcomings. For example, a coiled structure will do little to enhance the torqueability of a catheter, and a braided structure will do little to enhance the kink resistance of a catheter. Additionally, these structures occupy valuable space in the catheter. Since it is generally preferred to utilize a catheter having as small a diameter as possible for a particular use, the presence of any additional permanent structure in the catheter that may not be necessary for the intended use is inherently undesirable.

Another technique that has been developed to assist the physician in introducing a catheter into the vasculature is to construct a catheter to have a plurality of longitudinal sections that have varying durometers, or hardnesses. Generally, such catheters have a high durometer proximal section and one or more sections of decreasing durometer toward the distal end. In this manner, the catheter has greater hardness at the proximal end and greater flexibility at the distal end. However, this variation in durometer may result in a catheter having discrete sections of higher, or lower, hardness, flexibility, kink resistance and/or torqueability than is desired for a particular case. In addition, providing adjoining sections having different durometers may result in an undesirably abrupt transition of the catheter hardness from one section to another.

Catheters have also been developed that include a removable stiffening mandrel or like device that is utilized to assist in the insertion of the catheter. Frequently, the stiffening mandrel comprises an elongated rod-like structure that is inserted into a lumen of the catheter and extends along all, or a designated part, of the length of the catheter. The stiffening mandrel generally comprises a solid metal rod having a length that is intended to stiffen a commensurate length of the catheter. The distal portion of the metal rod may be ground to provide a gradual taper to the distal end of the rod, thereby enhancing the flexibility of the distal portion of the catheter relative to the proximal portion. Frequently, however, the flexibility of a catheter that incorporates a solid mandrel is less than desired. In addition, if the distal end is tapered as described, it may not retain sufficient strength to support the distal portion of the catheter. Finally, it can also be problematic to grind a rod-like metal to obtain the desired taper.

Therefore, it is desired to provide a stiffening member for use in a medical device, such as a catheter, that avoids the problems encountered with prior art devices. In addition, it is desired to provide a stiffening member that may be inserted into a lumen of a catheter, that provides a gradual transition from a less flexible section to a more flexible section, that provides support for the distal end of the catheter while maintaining enhanced pushability of the catheter, and that is effective for enhancing the kink resistance and/or torqueability of the catheter as the catheter is passed through narrow and/or tortuous areas of the vasculature of a patient.

### BRIEF SUMMARY

The problems of the prior art are addressed by the features of the present invention. In one form thereof, the present invention comprises a stiffening member for use in providing controllably variable stiffness to a catheter. The stiffening member is formed from an elongated tubular member sized to be received in a lumen of the catheter. At least a segment of the distal portion of the tubular member is configured, such as by forming a spiral cut therethrough, such that the distal portion of the tubular member has a greater flexibility than the proximal portion.

In another form thereof, the present invention comprises a catheter assembly comprising an elongated catheter shaft having at least one lumen therein, and a hollow tube sized to be received in the lumen. The tube is structured, such as by forming a spiral cut through a designated portion of the tube, in a manner such that the distal portion of the tube has a greater flexibility than the proximal portion of the tube.

### BRIEF DESCRIPTION OF THE DRAWINGS

FIG. 1 is a side view of a stiffening member in accordance with an embodiment of the present invention;

FIG. 2 is a side elevational view of a catheter assembly illustrating, in phantom, a stiffening member received in a lumen of the catheter;

FIG. 3 is an enlarged side view of a distal portion of the catheter assembly shown in FIG. 2, further illustrating a wire guide lumen in phantom;

FIG. 4 is an enlarged cross-sectional view along line 4-4 of FIG. 3; and

FIG. 5 is a cross-sectional view along line 5-5 of FIG. 3.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

The present invention relates to a stiffening member for use with a medical device, such as a catheter. In the following discussion, the terms “proximal” and “distal” will be used to describe the opposing axial ends of the stiffening member, as well as the axial ends of the medical device with which the stiffening member is used, and components thereof. The term “proximal” is used in its conventional sense to refer to the end of the member (or component) that is closest to the operator during use. The term “distal” is used in its conventional sense to refer to the end of the member (or component) that is initially inserted into the patient, or that is closest to the patient.

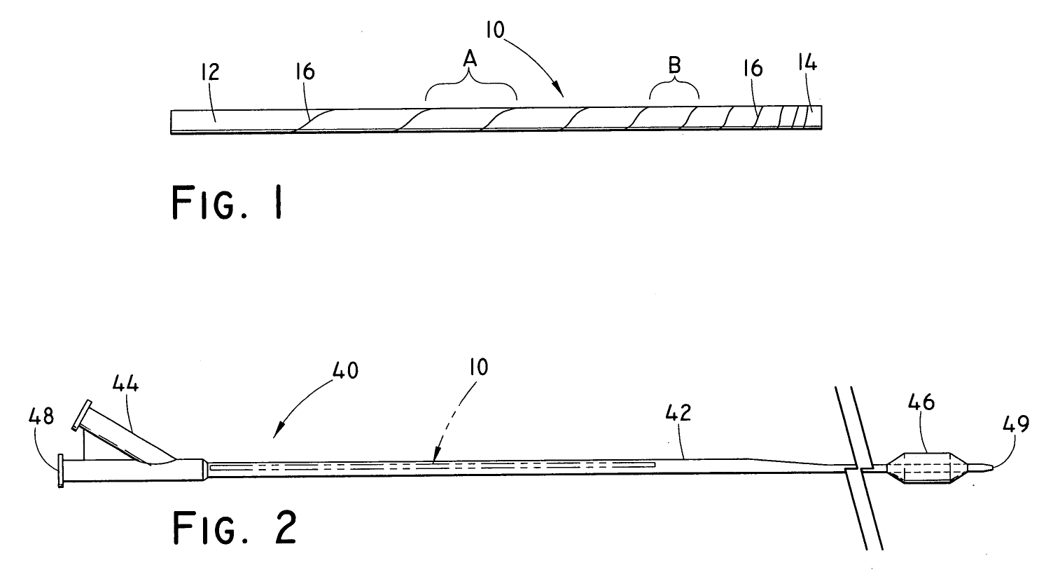

FIG. 1 is a side view of a stiffening member in accordance with an embodiment of the present invention. Stiffening member 10 comprises a hollow, elongated tubular member having a proximal portion 12, a distal portion 14, and a spiral cut 16 extending along at least a portion of the body of the stiffening member.

Stiffening member 10 is preferably formed of a metal or a metal alloy, or of a polymer or other composition having sufficient rigidity to impart a desired stiffness to a catheter or like device upon introduction of the catheter into the body of the patient. Non-limiting examples of typical compositions that may be used to form the stiffening member include stainless steel, nitinol, and relatively stiff polymers such as polytetrafluoroethylene (PTFE), high density polyethylene (HDPE) and polyether ether ketone (PEEK). Hollow elongated tubular materials (often referred to in the medical arts as “hypotubes”) that are suitable for use as a stiffening member are widely available from a number of commercial sources.

In the embodiment of FIG. 1, stiffening member 10 is spirally cut in a manner such that it has a relatively wide spacing between adjacent cuts in a proximal direction, and a relatively narrow spacing between adjacent cuts in a distal direction. This is illustrated in FIG. 1 wherein “A” denotes a space between adjacent cuts in proximal portion 12 of stiffening member 10, and “B” denotes a space between adjacent cuts in distal portion 14 of the stiffening member. Thus, as shown, length “A” is greater than length “B”. Portions having a wide spacing between adjacent cuts, such as spacing “A” in FIG. 1, are relatively stiff, whereas portions having a narrow spacing between adjacent cuts, such as spacing “B”, are relatively flexible. In the embodiment of FIG. 1, the spiral cut extends from distal portion of the stiffening member toward the proximal portion, but terminates prior to the proximal end. Since it is generally desired to have a relatively stiff proximal section, it will often be preferred to terminate the cut in this manner. However, if desired, the spiral cut may be extended to the proximal end of the stiffening member.

As shown in the preferred embodiment of FIG. 1, the spacing between adjacent spiral cuts becomes increasingly narrow in a gradual and continuous manner toward the distal end of the stiffening member. Thus, since the spacing between adjacent cuts gradually decreases in the distal direction, there will be only one length “A” and only one length “B” between adjacent cuts, with all other spacings between adjacent cuts being of a different length. Since the length between adjacent cuts decreases in the gradual and continuous manner described, the flexibility of the stiffening member increases in a gradual and continuous fashion toward the distal end of the tube. As a result, a smooth transition in the stiffness of the tubular member is provided from the stiffer proximal portion to the more flexible distal portion. Providing a gradual transition in this manner reduces the likelihood that a kink point will be created in the stiffening tube at a transition between a stiff, uncut, section and a section that has been spirally cut, or between adjoining cut sections wherein there is a significant difference in spacing between adjacent cuts in one section when compared to the adjoining section.

In one non-limiting example, a stiffening member comprises a spiral cut in the distal six inches of the tubular member. In this case, the tubular member has an outer diameter of about 0.012 inch (0.30 mm), and the spiral cut spacing reduces between adjacent cuts in a gradual and continuous manner from a maximum of about 0.10 inch (2.54 mm) to about 0.01 inch (0.254 mm) in the direction of the distal end. Preferably, a segment at the extreme distal end of the tubular member of about 0.02 inch (0.51 mm) remains uncut. Retaining a small uncut segment at the extreme distal end of the tubular member provides added strength to the extreme distal tip portion. A more proximal portion of the tubular member can include a spiral cut of greater than 0.10 inch (2.54 mm) between adjacent cuts if desired. Those skilled in that art will appreciate that the dimensions provided above represent only one example of a manner in which the spiral cuts may be formed in the elongated tubular member, and that other dimensions maybe selected when it is desired to provide more, or less, flexibility to a particular segment of the stiffening member.

The stiffening member 10 may have any length suitable for a particular use, and generally, will have a length similar to that of prior art solid metal stiffening members. Preferably, the stiffening member will have an inner diameter (ID) to outer diameter (OD) ratio of about 0.8 or less. A ratio of less than about 0.8 is common for metallic tubular members in order to minimize the likelihood that the tubular member will kink. When the ratio is above about 0.8, the tubular member has a greater likelihood of kinking, and also has a greater likelihood of maintaining its shape if bent, that is, it is subject to irreversible deformation upon bending. The compositions, dimensions, lengths and ratios of the stiffening member described hereinabove are only intended to represent examples of possible parameters. Those skilled in the art will appreciate that other compositions, dimensions, lengths and ratios may be suitable for a particular application, which parameters may be selected utilizing no more than routine experimentation when the teachings of the present application are applied.

FIG. 2 is a side view of a catheter assembly 40. Catheter assembly 40 comprises a catheter shaft 42, and may also include other conventional features and/or attachments, such as the conventional side arm 44 at its proximal end and a conventional balloon at its distal end. Catheter side arms are used for many well-known purposes, such as the introduction or aspiration of a fluid to or from the vasculature. Catheter balloon 46 may be used, e.g., for dilating a vessel during a percutaneous transluminal coronary angioplasty (PTCA) procedure. Although catheter assembly 40 is shown herein as a balloon catheter assembly, this depiction is merely for the purpose of describing an exemplary structure from which to illustrate the features of the invention, and not by way of limitation. The teachings of the present invention are applicable to any type of medical device which would be benefited by the incorporation of a stiffening member having a variable stiffness along its length, regardless of whether the device comprises a catheter and/or whether it includes a balloon or other known structure. As illustrated in phantom in FIG. 2, a stiffening member 10 is received in a lumen of catheter shaft 42.

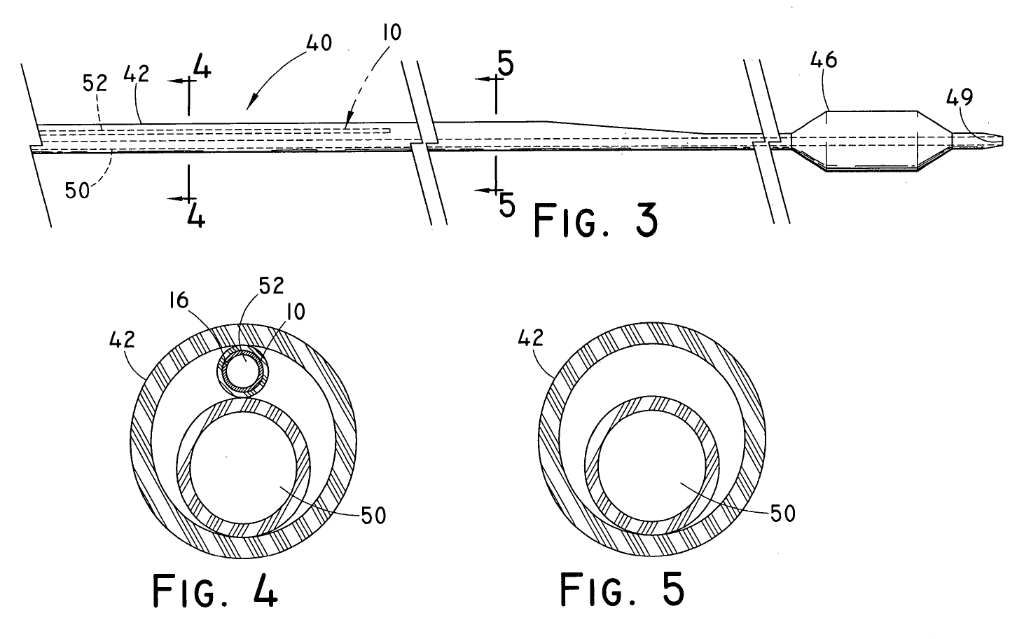

FIG. 3 is an enlarged side view of a distal portion of the catheter assembly shown in FIG. 2. Catheter assembly lumens 50, 52 are shown in phantom. Lumen 50 extends the length of catheter shaft 42, from open proximal end 48 to open distal end 49 (FIG. 2), and is typically dimensioned to receive a wire guide. Lumen 52 extends from open proximal end 48 to a designated terminal portion within shaft 42, and is dimensioned to receive the stiffening member.

FIG. 4 is an enlarged cross-sectional view taken along line 4-4 of FIG. 3. This figure illustrates the positioning of lumens 50, 52 in catheter shaft 42. Stiffening member 10 is also shown in lumen 52. FIG. 5 is an enlarged cross-sectional view taken along line 5-5 of FIG. 3. Line 5-5, taken from a more distal point of catheter shaft 42, illustrates the presence of lumen 50, and also indicates the absence of lumen 52 (and stiffening member 10) in the most distal portion of the catheter shaft.

The spiral cut in the stiffening member may be accomplished by any conventional method for making cuts in tubular materials. In order to provide optimal control, it is preferred to form the spiral cuts by utilizing a computer-controlled and driven cutting means, such as a laser. The use of a computer-controlled and driven laser enables the operator to very carefully control these parameters over a desired length of the stiffening member. Laser cutting of medical tubes is a well-known technique, and those skilled in the art can readily adapt this technique to a particular application without undue experimentation. Although computer-controlled drivers are preferred, such cutting means may also be manually controlled and driven. As an alternative to the use of lasers and the like, a stiffening member may also be cut by known manual means, such as by scoring the tube as it is rotated by a lathe. It is normally preferred to make the spiral cut such that it extends fully through the wall of the tube. However, this is not required, and the spiral cut can be made partially through the wall of the tubular member in the nature of a scored line.

Once a tubular member has been spirally cut as described above, the flexibility of the resulting stiffening member can be even further modified by combining the spiral cut with one or more known flexibility techniques. For example, flexibility of the spiral cut stiffening member can be further adjusted by varying the wall diameters (ID and/or OD) of the spiral cut tubular member. Thus, a selected portion of these diameters (typically the distal portion) can be ground, etched, tapered and/or otherwise altered in a known manner to reduce the diameter of this portion compared to a non-altered portion, thereby varying the flexibility along the shaft of the tubular member. Furthermore, the grinding, etching, etc., of the stiffening member can be gradually increased and/or decreased as desired to provide a smooth transition in flexibility along the length of the stiffening member.

Another technique that may be used to further vary the flexibility of the stiffening member is to combine a conventional rod-like stiffening mandrel with the spiral cut tubular member. For example, a stiffening mandrel can be sized such that it is receivable in the lumen of hollow stiffening member 10. The stiffening mandrel can have a length such that it only imparts rigidity along a designated length of the stiffening member, or along the entire length. Furthermore, the distal portion of the stiffening mandrel can be tapered in a conventional manner if desired, to provide additional flexibility. The use of a stiffening mandrel in combination with the spiral cut stiffening member offers much latitude for the amount of stiffness desired, particularly at the non-spiral cut proximal end. When a conventional stiffening mandrel is utilized, it is preferred to use a mandrel having the same basic composition as the tubular member, in order to reduce the possibility of corrosion. However, if desired, either of these elements can be varied in size, shape, and/or composition in a particular case to achieve an optimal level of stiffness or flexibility. For example, if only a small amount of additional stiffness is desired, a stiffening mandrel formed of a less stiff material, such as nitinol, can be utilized with the stiffening member. If a greater amount of stiffness is desired, the stiffening rod can be formed of a more rigid material, such as a stainless steel formulation.

Another technique that may be used to further vary the flexibility of the stiffening member is to incorporate a rigid elongated structure, such as a polymer bead, inside the structure of the tubular member along all or a portion of its length. In addition to providing enhanced flexibility at a desired length of the stiffening member, the bead provides enhanced support to the stiffening member. The bead can be positioned at varied depths along the shaft, or alternatively, can be tapered to provide greater latitude in flexibility.

Yet another technique for varying the flexibility of the stiffening member is to provide a coating over all, or a portion, of the inner or outer surface of the stiffening member. A non-limiting list of suitable coating agents includes nylon, polyurethane and PEBA (polyether block amide). Providing a coating of such materials on the stiffening member varies the stiffness or flexibility of the coated portion of the stiffening member relative to the uncoated portion. The variations of the selected segments may be accomplished, e.g., by applying the coating to a designated segment of the tube, by applying more, or thicker, coating to a designated segment relative to another segment, or by applying different coatings to different portions of the tube. Other coating methods may include, for example, dip coating, over-extrusion of the polymeric coating onto the stiffening member, and heat shrinking the coating as a laminate onto the stiffening member. This could be done in a conventional heat shrink envelope, such as one formed of PET, PEBA or polyolefin. Those skilled in the art are well aware of suitable coating methods, and other known coating methods not specifically mentioned herein may also be suitable for use in the present invention. In addition to the foregoing, coatings may also be applied for other known reasons, such as to vary the lubricity of a designated segment relative to another segment.

It is believed that the use of a spiral cut tubular member provides greater support when compared to a conventional solid stiffening mandrel, thereby enhancing the kink resistance and/or the pushability of the catheter in which the stiffening member is inserted. Similarly, it is believed that the use of a spiral cut tubular member also provides greater latitude in flexibility control when compared to the solid mandrel.

Although each of the embodiments described hereinabove discloses a tubular member having a continuous spiral cut formed therein, this need not necessarily be the case. Rather, in some instances, instead of cutting a continuous spiral along a length of the tubular member, the flexibility of a tubular member may be modified by making a cut in a designated portion of the tube, and not in another designated portion. Similarly, a cut may be made in other than spiral fashion along a designated portion of the tube. Still further, the cut need not be continuous, but rather, may be discontinuous along a length of the tube. In each of these alternatives, the flexibility of a designated portion of a tube will be modified. Similarly, those skilled in the art will appreciate that it is not always necessary to form any cuts in the tubular member. Rather, the flexibility of the tubular member can be modified by varying the wall diameters (ID and/or OD) as described above, for example, by grinding, etching, tapering and/or by otherwise altering the tube in a known manner to vary the flexibility along a length of the tubular member, and/or by applying a coating to at least a portion of the tubular member.

Since the inventive stiffening member can have the same dimensions as a conventional solid stiffening mandrel, the inventive stiffening member can be easily substituted for conventional mandrels or rods of the type that are presently in common use for such purpose. Thus, the same catheters that are commonly used with known stiffening mandrels, such as PTCA catheters, can continue to be used with the inventive stiffening members. The inventive stiffening members can be advantageously used with conventional over-the-wire (OTW) catheters, as well as with rapid exchange (RX) catheters.

It is therefore intended that the foregoing detailed description be regarded as illustrative rather than limiting, and that it be understood that it is the following claims, including all equivalents, that are intended to define the spirit and scope of this invention.

1. A stiffening member for use in providing controllably variable stiffness to a catheter, comprising:
an elongated tubular member sized to be received in a lumen of said catheter, said tubular member having a proximal portion and a distal portion, wherein at least a length of said distal portion is configured such that said distal portion length has a greater flexibility than a length of said proximal portion.

2. The stiffening member of claim 1, wherein at least said distal portion length is configured by forming a generally spiral cut therethrough.

3. The stiffening member of claim 2, wherein said generally spiral cut extends along at least a portion of said proximal portion length and said distal portion length.

4. The stiffening member of claim 2, wherein a spacing between adjacent cuts of said spiral cut decreases toward the distal end of said tubular member.

5. The stiffening member of claim 4, wherein said spacing decreases in a continuous manner toward said distal end of said tubular member.

6. The stiffening member of claim 5, wherein said spacing decreases from a distance of about 0.10 inch between adjacent windings of said spiral cut to a distance of about 0.01 inch between adjacent windings at said distal end.

7. The stiffening member of claim 6, wherein said spacing decrease occurs over a length of about the distal-most six inches of the tube.

8. The stiffening member of claim 6, wherein a terminal distal end portion of said tubular member remains uncut, said uncut portion comprising a length of about 0.02 inch.

9. The stiffening member of claim 1, wherein said tubular member has an inner diameter and an outer diameter, and wherein the ratio of the inner diameter to the outer diameter does not exceed about 0.8.

10. The stiffening member of claim 1, wherein said tubular member comprises a composition selected from the group consisting of stainless steel, nitinol, PTFE, HDPE and PEEK.

11. The stiffening member of claim 1, wherein said tubular member distal portion is configured by tapering at least a segment of a distal wall portion from a larger diameter to a smaller diameter in a distal direction.

12. The stiffening member of claim 2, wherein at least a portion of said distal portion length is tapered from a larger diameter to a smaller diameter in a distal direction.

13. The stiffening member of claim 1, wherein at least a portion of said tubular member is provided with a coating for varying said flexibility.

14. The stiffening member of claim 2, wherein at least a portion of said tubular member is provided with a coating for varying said flexibility.

15. A catheter assembly, comprising:
an elongated catheter shaft having at least one lumen therein; and
a hollow tube sized to be received in said lumen, said tube having a proximal portion and a distal portion, said tube being structured such that a distal portion of said tube has a greater flexibility than a proximal portion of said tube.

16. The catheter assembly of claim 15, wherein at least said distal portion of said tube has a generally spiral cut extending therealong.

17. The catheter assembly of claim 16, wherein a spacing between adjacent turns of said spiral cut decreases in a direction of said distal end.

18. The catheter assembly of claim 17, wherein said spacing decreases in a continuous manner toward said distal end.

19. The catheter assembly of claim 17, wherein at least a segment of a distal wall portion is tapered from a larger diameter to a smaller diameter in a distal direction.

20. The catheter assembly of claim 17, wherein at least a portion of said tube is provided with a coating for varying said flexibility.
